(19) United States
(12) Patent Application Publication
(10) Pub. No.: US 2026/0315518 A1
(43) Pub. Date: Oct. 15, 2026

(54) METHOD FOR EXTRACTING ALGAL POLYSACCHARIDE FOR WHOLE-ALGAE UTILIZATION, AND USES OF THE EXTRACTED ALGAL POLYSACCHARIDE

(71) Applicants: BO-RUI CHEN, Taichung City (TW); PINJIA Biotech Co., Ltd., Donggang Township (TW)

(72) Inventors: BO-RUI CHEN, Taichung City (TW); HSIN-CHUN HSU, Donggang Township (TW)

(21) Appl. No.: 19/641,178

(22) Filed: Apr. 7, 2026

(30) Foreign Application Priority Data
Apr. 9, 2025 (TW) 114113397

(51) Int. Cl. A61K 31/715; A61P 1/14; C08B 37/00

(52) U.S. Cl. CPC A61K 31/715; A61P 1/14; C08B 37/006

(57) ABSTRACT

A method for extracting an algal polysaccharide for whole-algae utilization is disclosed, along with uses of the extracted algal polysaccharide. The disclosed method for extracting an algal polysaccharide for whole-algae utilization can be used to produce an algal polysaccharide having a high polysaccharide content and prebiotic activity from an algal raw material, while yielding algal co-products through a multi-stage extraction procedure. Thus, the disclosed method for extracting an algal polysaccharide for whole-algae utilization can not only produce a special algal polysaccharide for use in health supplements or skincare products, but also greatly reduce extraction waste to contribute to environmental sustainability.

### FIELD OF THE INVENTION

The present invention relates to an algal extraction technique and applications of the extracted products. More particularly, the invention relates to a method for extracting an algal polysaccharide for whole-algae utilization, and uses of the extracted algal polysaccharide.

### DESCRIPTION OF THE RELATED ART

Seaweed, also referred to as macroalgae, refers to multicellular algae that grow in the ocean and are visible to the naked eye. Some common examples are green algae, brown algae, red algae, and blue-green algae (also known as cyanobacteria). The thallus of seaweed contains a diverse array of nutrients, such as essential amino acids for the human body, proteins, vitamins, minerals, and polysaccharides. Recent studies have further pointed out that seaweed has various health-maintaining and cosmetic benefits. Therefore, seaweed has been widely used in food products, health supplements, and skincare products, among others.

To obtain the active ingredients of seaweed, it is common practice nowadays to extract the desired ingredients from seaweed by an extraction technique, e.g., by using methanol, ethyl acetate, and n-butanol as extraction solvents to isolate algal polysaccharides from the thallus of seaweed. However, while conventional extraction techniques are capable of deriving the desired ingredients, they still have the following deficiencies.

First, the extraction process is operationally complex as a whole, requires multiple organic solvents, does not support continuous extraction, and therefore results in high production costs.

Second, a large amount of extraction waste is generated from the extraction process. This not only adds to a manufacturer's waste disposal costs but also makes it difficult to achieve sustainability and environmental protection.

### SUMMARY OF THE INVENTION

The primary objective of the present invention is to provide a method for extracting an algal polysaccharide for whole-algae utilization, and uses of the extracted algal polysaccharide. The algal polysaccharide extracted by the method disclosed in the invention has a high polysaccharide content and a unique monosaccharide composition, exhibits anti-allergic, intestinal microbiota-regulating, anti-inflammatory, and wound repair-promoting activities, and therefore can be used as a primary active ingredient in food products or skincare products.

Another objective of the present invention is to provide a method for extracting an algal polysaccharide for whole-algae utilization, and uses of the extracted algal polysaccharide, wherein the method disclosed in the invention for extracting an algal polysaccharide can yield useful algal co-products through a multi-stage extraction procedure, thereby effectively reducing the generation of extraction waste and being environmentally friendly.

To achieve the foregoing objectives, the present invention provides a method for extracting an algal polysaccharide for whole-algae utilization, and uses of the extracted algal polysaccharide. The algal polysaccharide produced by the method disclosed in the invention has a high polysaccharide content and exhibits prebiotic, anti-allergic, anti-inflammatory, wound repair-promoting, and anti-ultraviolet (anti-UV) activities. Therefore, administering an effective amount of the algal polysaccharide disclosed in the invention or a composition containing the algal polysaccharide to an individual can regulate the microbiota in the individual's intestinal tract and effectively produce such effects as preventing or ameliorating diseases associated with inflammation and diseases associated with intestinal dysbiosis, resisting skin aging, and promoting wound repair.

The algal polysaccharide has a total sugar content of at least 80 (w/w) %, a water-soluble component of at least 70 (w/w) %, and a viscosity of 2200-2510 cps. The monosaccharide composition of the algal polysaccharide includes 56-63% galactose, 27-33% glucose, 5-6% xylose, and 3-5% mannose.

In one embodiment of the present invention, the total sugar content of the algal polysaccharide is at least 90 (w/w) %.

In one embodiment of the present invention, the method for extracting an algal polysaccharide for whole-algae utilization includes the following three-stage extraction procedure, in which the first-stage through the third-stage extraction processes are performed in a sequential and continuous manner. The first-stage extraction process involves performing extraction on an algal raw material with a first extraction solvent to obtain a first extraction mixture. The second-stage extraction process involves collecting a first precipitate from the first extraction mixture and performing extraction on the first precipitate with a second extraction solvent to obtain a second extraction mixture. The third-stage extraction process involves collecting a second precipitate from the second extraction mixture, performing extraction on the second precipitate with a third extraction solvent to obtain a third extraction mixture, and filtering the third extraction mixture to obtain a third filtrate, wherein the third filtrate contains an algal polysaccharide having a high polysaccharide content.

The second extraction solvent used in the second-stage extraction process is different from the first and the third extraction solvents used in the first-stage and the third-stage extraction processes, and the extraction temperatures of the first-stage and the second-stage extraction processes are lower than the extraction temperature of the third-stage extraction process.

In another embodiment of the present invention, the extraction temperature of each of the first-stage and the second-stage extraction processes is 10-40° C., whereas the extraction temperature of the third-stage extraction process is 90-100° C.

In yet another embodiment of the present invention, the first and the third extraction solvents used in the first-stage and the third-stage extraction processes are water, whereas the second extraction solvent used in the second-stage extraction process is a lower alcohol such as ethanol.

In addition, the multi-stage extraction procedure of the disclosed method for extracting an algal polysaccharide for whole-algae utilization yields algal co-products for industrial use, thereby achieving whole-algae utilization, environmental friendliness, and economic value enhancement. More specifically, the first-stage extraction process yields an extract containing phycobiliproteins, the second-stage extraction process yields an extract containing phytochemicals, and the third-stage extraction process yields an extract containing an algal polysaccharide and an extract containing seaweed fiber.

In still another embodiment of the present invention, the algal polysaccharide produced by the disclosed method for extracting an algal polysaccharide for whole-algae utilization can be used to prepare a composition for regulating the intestinal microbiota, a composition for resisting UV damage, or a cytokine inhibitor.

The aforesaid compositions and inhibitor may each be a food product, a skincare product, or a nutritional supplement.

When the algal polysaccharide is used in the preparation of a composition for regulating the intestinal microbiota, the resulting composition for regulating the intestinal microbiota can be used to regulate the growth of beneficial bacteria and harmful bacteria in the intestinal tract, thereby achieving a balanced intestinal microbiota. More specifically, the composition for regulating the intestinal microbiota can increase the abundance of intestinal microbiota classified under the family Bifidobacteriaceae, Lactobacilluseae, Muribaculaceae, or Bacteroidaceae and elevate the short-chain fatty acid levels in the intestinal tract, while inhibiting the growth of microorganisms classified under the family Clostridiaceae or Enterobacteriaceae.

When the algal polysaccharide is used in the preparation of a composition for resisting UV damage, the resulting composition for resisting UV damage can be used to enhance the skin barrier function, thereby preventing skin damage or aging caused by UV irradiation.

When the algal polysaccharide is used in the preparation of a cytokine inhibitor, the resulting cytokine inhibitor can be used to inhibit the concentration of IL-4, IL-6, IL-8, or histamine, thereby achieving an anti-allergic and/or anti-inflammatory effect.

When the algal polysaccharide is used in the preparation of a composition for promoting wound repair, the resulting composition for promoting wound repair can be used to increase the expression level of the MMP-9, VEGF-A, or KRT16 gene and promote epithelial cell migration, thereby achieving the effect of promoting wound healing and repair.

### BRIEF DESCRIPTION OF DRAWINGS

FIG. 1 is the flowchart of the method in the first embodiment of the present invention for extracting an algal polysaccharide for whole-algae utilization.

FIG. 2 shows the result of a Fourier-transform infrared (FTIR) analysis of the first batch of the algal polysaccharide disclosed in the present invention.

FIG. 3 shows the relative abundance of Bifidobacteriaceae in the intestinal tract of the mice in each group.

FIG. 4 shows the relative abundance of Lactobacilluseae in the intestinal tract of the mice in each group.

FIG. 5 shows the relative abundance of Muribaculaceae in the intestinal tract of the mice in each group.

FIG. 6 shows the relative abundance of Bacteroidaceae in the intestinal tract of the mice in each group.

FIG. 7 shows the relative abundance of Clostridiaceae in the intestinal tract of the mice in each group.

FIG. 8 shows the relative abundance of Enterobacteriaceae in the intestinal tract of the mice in each group.

FIG. 9 shows the distribution of intestinal microbiota in the mice in each group.

FIG. 10 shows the acetate concentrations of the mice in each group before and after the experiment.

FIG. 11 shows the propionate concentrations of the mice in each group before and after the experiment.

FIG. 12 shows the butyrate concentrations of the mice in each group before and after the experiment.

FIG. 13 shows the IL-4 concentrations in P815 cells treated with Sarcodia suae polysaccharide at different concentrations.

FIG. 14 shows the histamine concentrations in P815 cells treated with Sarcodia suae polysaccharide at different concentrations.

FIG. 15 shows the IL-6 concentrations in RAW264.7 cells treated with Sarcodia suae polysaccharide at different concentrations.

FIG. 16 shows the IL-8 concentrations in RAW264.7 cells treated with Sarcodia suae polysaccharide at different concentrations.

FIG. 17 shows the fluorescence responses of HaCaT cells treated with Sarcodia suae polysaccharide at different concentrations.

FIG. 18 shows the analysis result of the fluorescence areas of HaCaT cells treated with Sarcodia suae polysaccharide at different concentrations.

FIG. 19 shows the cell proliferation rates of HaCaT cells treated with Sarcodia suae polysaccharide at different concentrations.

FIG. 20 shows the expression levels of VEGF-A in HaCaT cells treated with Sarcodia suae polysaccharide at different concentrations.

FIG. 21 shows the expression levels of MMP-9 in HaCaT cells treated with Sarcodia suae polysaccharide at different concentrations.

FIG. 22 shows the expression levels of KRT-16 in HaCaT cells treated with Sarcodia suae polysaccharide at different concentrations.

### DETAILED DESCRIPTION OF THE INVENTION

The present invention discloses a method for extracting an algal polysaccharide for whole-algae utilization, and uses of the extracted algal polysaccharide. The disclosed method for extracting an algal polysaccharide for whole-algae utilization includes a three-stage extraction procedure composed essentially of a first-stage extraction process, a second-stage extraction process, and a third-stage extraction process, wherein the different stages use different extraction solvents and conditions to derive an algal polysaccharide with a high polysaccharide content, along with algal co-products, from an algal raw material. The algal polysaccharide produced by the disclosed extraction method has diverse bioactivities, such as intestinal microbiota-regulating, UV damage-resisting, anti-inflammatory, and anti-allergic activities. Therefore, administering an effective amount of the algal polysaccharide to an individual can achieve such effects as ameliorating diseases associated with allergy, alleviating inflammation or diseases associated therewith, mitigating diseases associated with intestinal dysbiosis, resisting skin aging, and promoting wound repair.

More specifically, the algal polysaccharide disclosed in the present invention has a total sugar content higher than 80 (w/w) %, a water-soluble component of at least 70 (w/w) %, and a viscosity of 2200-2510 cps. The primary monosaccharides of the algal polysaccharide are galactose (56-63%), glucose (27-33%), xylose (5-6%), and mannose (0.5-2%). Other sugars account for approximately 3-5%.

The extraction solvent used in the second-stage extraction process is different from the extraction solvents used in the first-stage and the third-stage extraction processes. For example, the first-stage and the third-stage extraction processes each use water as the extraction solvent, whereas the second-stage extraction process uses a lower alcohol, such as ethanol at a concentration of 75-90%, as the extraction solvent. Or, the first-stage and the third-stage extraction processes each employ solvent extraction as the extraction method and water as the extraction solvent, whereas the second-stage extraction process employs supercritical fluid extraction as the extraction method and carbon dioxide as the extraction solvent.

The extraction temperature of each of the first-stage extraction process and the second-stage extraction process is 10-40° C., and the extraction temperature of the third-stage extraction process is higher than those of the first-stage and the second-stage extraction processes. For instance, the extraction temperature of the first-stage extraction process is 20-40° C., such as 20° C., 25° C., 30° C., 35° C., or 40° C.; the extraction temperature of the second-stage extraction process is 10-30° C., such as 10° C., 15° C., 18° C., 20° C., 25° C., 28° C., or 30° C.; and the extraction temperature of the third-stage extraction process is 90-100° C., such as 90° C., 95° C., 98° C., or 100° C.

The algal co-products yielded by the multi-stage extraction procedure of the method of the present invention for extracting an algal polysaccharide for whole-algae utilization can be used in different industries or different products, thereby achieving the effects of whole-algae utilization, economic value enhancement, and environmental sustainability.

More specifically, the first-stage extraction process yields an extract containing phycobiliproteins, the second-stage extraction process yields an extract containing phytochemicals, and the third-stage extraction process yields seaweed fiber as well as the algal polysaccharide disclosed in the present invention.

The crude product obtained from each stage of the extraction procedure may be subjected to a drying treatment to facilitate transportation and storage. The drying treatment may be any moisture removal technique commonly used by a person of ordinary skill in the technical field to which the present invention pertains, such as freeze drying, spray drying, or hot air drying.

Unless otherwise stated, the scientific terms used in this description and the appended claims shall be interpreted according to textbooks, dictionaries, or general common knowledge in the technical field to which the present invention pertains.

The term “algal raw material” refers to a material obtained by washing, pulverizing, crushing, cutting, drying, or otherwise processing seaweed, wherein the seaweed includes red algae, brown algae, and so on. One example of such an algal raw material is algal powder.

The term “Sarcodia suae” refers to a large red alga growing in the ocean.

The term “phycobiliproteins” refers to water-soluble proteins, or protein complexes, present in algae.

The term “phytochemicals” refers to essential elements for algal growth that provide color, aroma, and self-protection capabilities (e.g., the ability to remove peroxide free radicals) to the algae.

The term “seaweed fiber” refers to fiber isolated from seaweed.

The term “composition” refers to a mixture containing the algal polysaccharide disclosed in the present invention, wherein the mixture may be a skincare product, a food product, or a nutritional supplement, for example. In cases where the composition is a food product, the food product contains an ingredient acceptable in the food product industry, in addition to the algal polysaccharide disclosed in the invention, and the food product may be manufactured in various forms such as drinks, cookies, and jelly. In cases where the composition is a skincare product, the skincare product includes a carrier and/or an excipient acceptable in the skincare product industry, in addition to the disclosed algal polysaccharide as a main active ingredient. In cases where the composition is a nutritional supplement, the nutritional supplement includes a carrier and/or an excipient acceptable in the nutritional supplement industry, in addition to the disclosed algal polysaccharide as a main active ingredient.

The term “active ingredient” refers to an ingredient capable of achieving the claimed efficacy or purpose. For example, if A is an active ingredient of a composition for reducing blood glucose levels, A has the activity of lowering or stabilizing blood glucose levels.

The term “metabolic disease,” as used in the present invention, refers to a disease associated with intestinal dysbiosis, such as hyperglycemia, hyperlipidemia, or obesity caused by intestinal dysbiosis.

The term “disease associated with intestinal dysbiosis” refers to a disease caused by an imbalance where the population of harmful bacteria in the intestinal tract exceeds that of beneficial bacteria. According to recent studies, diseases associated with intestinal dysbiosis include depression, Alzheimer's disease, and immune-related diseases, among others.

Each numerical value disclosed herein shall be interpreted as including the disclosed numerical value and the same value plus or minus an error acceptable in the technical field to which the present invention pertains, such as 10% of the disclosed value. For instance, 20° C. as disclosed herein shall be interpreted as 20±2° C.

To expound the technical features of the present invention, an embodiment is provided and detailed below.

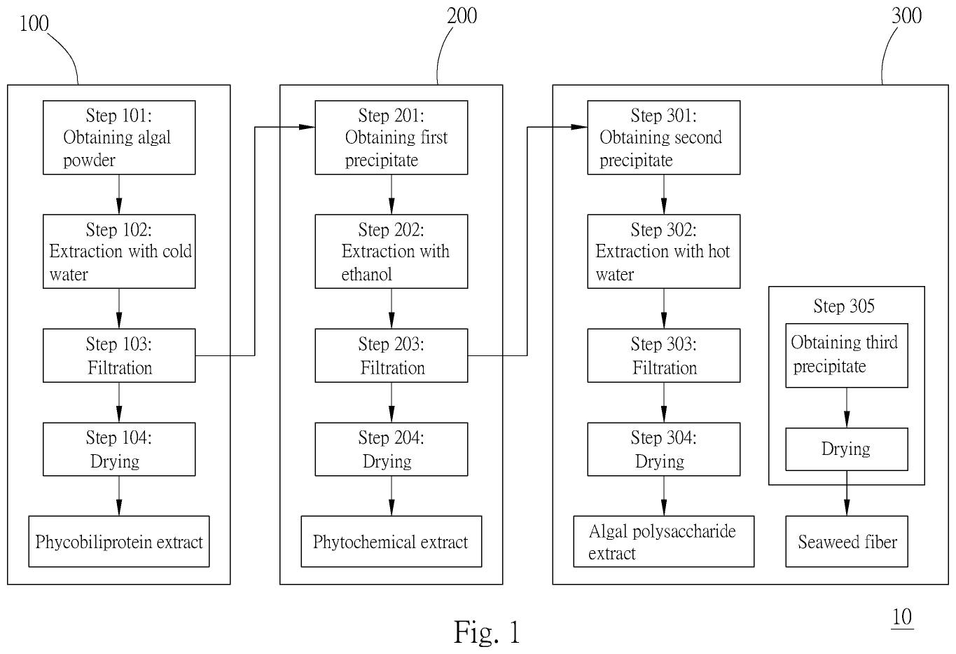

Referring to FIG. 1, the first embodiment of the present invention discloses a method 10 for extracting an algal polysaccharide for whole-algae utilization. The method 10 can be used to derive an algal polysaccharide from an algal raw material and includes a first-stage extraction process 100, a second-stage extraction process 200, and a third-stage extraction process 300.

The first-stage extraction process 100 includes the following steps:

Step 101: Sarcodia suae algal powder is obtained. The Sarcodia suae algal powder can be purchased from a commercial source or obtained by subjecting Sarcodia suae sequentially to washing, air drying (at 40° C.), and crushing.

Step 102: The Sarcodia suae algal powder is subjected to extraction with cold water for 30-60 minutes to yield a first extraction mixture. The weight ratio of the Sarcodia suae algal powder to the cold water is 1:20-30, and the extraction temperature is 20-40° C.

Step 103: The first extraction mixture is filtered to yield a first filtrate and a first precipitate.

Step 10⁴: The first filtrate is collected, and a phycobiliprotein extract is obtained by removing water from the first filtrate using a freeze-drying technique.

The second-stage extraction process 200 is performed subsequent to the first-stage extraction process 100 and includes the following steps:

Step 201: The first precipitate is collected.

Step 202: The first precipitate is subjected to extraction with ethanol at a concentration of 75-95% to yield a second extraction mixture. The weight ratio of the first precipitate to the ethanol is 1:20-30, and the extraction temperature is 10-30° C.

Step 203: The second extraction mixture is filtered to yield a second filtrate and a second precipitate.

Step 204: The second filtrate is collected, and a phytochemical extract is obtained by removing water from the second filtrate using a freeze-drying technique.

The third-stage extraction process 300 is performed subsequent to the second-stage extraction process 200 and includes the following steps:

Step 301: The second precipitate is obtained.

Step 302: The second precipitate is subjected to extraction with hot water for 60-120 minutes to yield a third extraction mixture. The weight ratio of the second precipitate to the hot water is 1:30-50, and the extraction temperature is approximately, or equal to, 100° C.

Step 303: The third extraction mixture is filtered to yield a third filtrate and a third precipitate.

Step 304: The third filtrate is collected, and an algal polysaccharide extract (which has a brown color) is obtained by removing water from the third filtrate using a spray-drying technique.

Step 305: The third precipitate is collected, and seaweed fiber is obtained by removing water from the third precipitate using a hot air-drying technique.

The filtering step in each of the foregoing stages can be preceded by checking the flow rate, moisture content, weight, and so on to ensure extraction is complete.

In the method in another embodiment of the present invention for extracting an algal polysaccharide for whole-algae utilization, the steps are generally the same as those in the aforesaid embodiment, except that the extraction technique used in the second-stage extraction process is supercritical fluid extraction, meaning that the second extraction mixture is obtained by extraction with carbon dioxide as the extraction solvent.

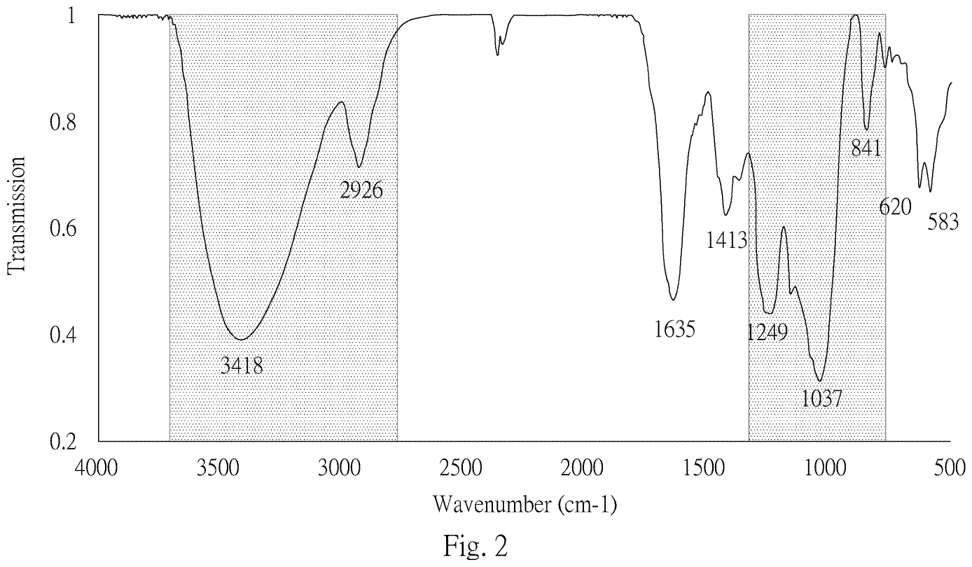

The extraction method stated above was used to yield different batches of algal polysaccharide extracts. These batches were analyzed for polysaccharide content, viscosity, and monosaccharide composition and were further characterized by Fourier-transform infrared (FTIR) spectroscopy. The results are shown in Table 1 to Table 3 and FIG. 2. Viscosity was determined by the Brookfield viscosity measurement method.

TABLE 1
Monosaccharide composition analysis of
different algal polysaccharide batches

| Monosaccharide | | | |
|---|---|---|---|
| composition | First batch | Second batch | Third batch |
| Galactose | 58.61% | 62.54% | 56.96% |
| Glucose | 29.89% | 27.32% | 32.15% |
| Xylose | 5.85% | 5.12% | 6.31% |
| Mannose | 0.93% | 1.92% | 0.50% |
| Other sugars | 4.70% | 3.10% | 4.10% |

TABLE 2
Viscosity analysis of different algal polysaccharide batches

| | Viscosity (cps) |
|---|---|
| First batch | 2430 |
| Second batch | 2200 |
| Third batch | 2510 |

TABLE 3
Polysaccharide property analysis of different algal polysaccharide batches

| Property | First batch | Second batch | Third batch |
|---|---|---|---|
| Solid weight of sample after drying | 1 g | 0.84 g | 1.1 g |
| Water-soluble component (water- | 76 (w/w)% | 81 (w/w)% | 70 (w/w)% |
| soluble dietary fiber content) | | | |
| Total sugar content | 90 (w/w)% | 83 (w/w)% | 92 (w/w)% |
| Free sugar content | 0 | 0 | 3 |

According to the analysis results presented above, the algal polysaccharide obtained by the extraction method disclosed in the present invention has a polysaccharide content of 83-92 (w/w) %, a water-soluble component of 70-81 (w/w) %, and a viscosity of 2200-2510 cps. The main monosaccharides in the algal polysaccharide were galactose, glucose, xylose, and mannose, which were present at 56-63%, 27-33%, 5-6%, and 0.5-2%, respectively, with other sugars accounting for about 3-5%.

It can be further known from the results in FIG. 2 that the algal polysaccharide disclosed in the present invention exhibited a strong and broad signal in the range of 3600-3000 cm⁻¹, along with sulfate-group signals at 1249, 1037, and 841 cm⁻¹. This indicates that the disclosed algal polysaccharide is composed of α-configuration sugar units and is rich in sulfate groups.

In addition, the algal polysaccharide disclosed in the present invention was subjected to heavy metal testing and in vitro cytotoxicity assays. The results indicate that the algal polysaccharide obtained by the extraction method disclosed in the invention complied with heavy metal safety standards and had no cytotoxicity.

From the description of the composition of the steps stated above, it can be known that the method disclosed in the present invention for extracting an algal polysaccharide for whole-algae utilization uses a continuous extraction technique to safely and rapidly isolate a galactose-rich algal polysaccharide and other algal co-products from an algal raw material. This method significantly reduces extraction waste generated during the extraction processes and yields a high-purity, highly bio-safe algal polysaccharide through a specific multi-stage extraction procedure, thereby enhancing the economic value of algae and lowering production costs.

The second embodiment of the present invention includes substantially the same steps as in the first embodiment, except that in step 305, bleaching is performed before the third precipitate is dried, the objective being to obtain seaweed fiber with a consistent color, thereby increasing the possibility of subsequent application.

To verify the combined bioactivities of the algal polysaccharide disclosed in the present invention, several examples are provided below and detailed with reference to the accompanying drawings.

The Sarcodia suaep olysaccharide used in the following examples was prepared according to the disclosure of the first embodiment.

### Example 1: Intestinal Microbiota Analysis

A plurality of six-week-old BALB/c mice were randomly divided into three groups, each treated according to the corresponding conditions below for 14 days:
Group 1: Daily administration of reverse osmosis (RO) water.
Group 2: Daily oral administration of Sarcodia suae polysaccharide at 20 mg/kg/day.
Group 3: Daily oral administration of Sarcodia suae polysaccharide at 40 mg/kg/day.

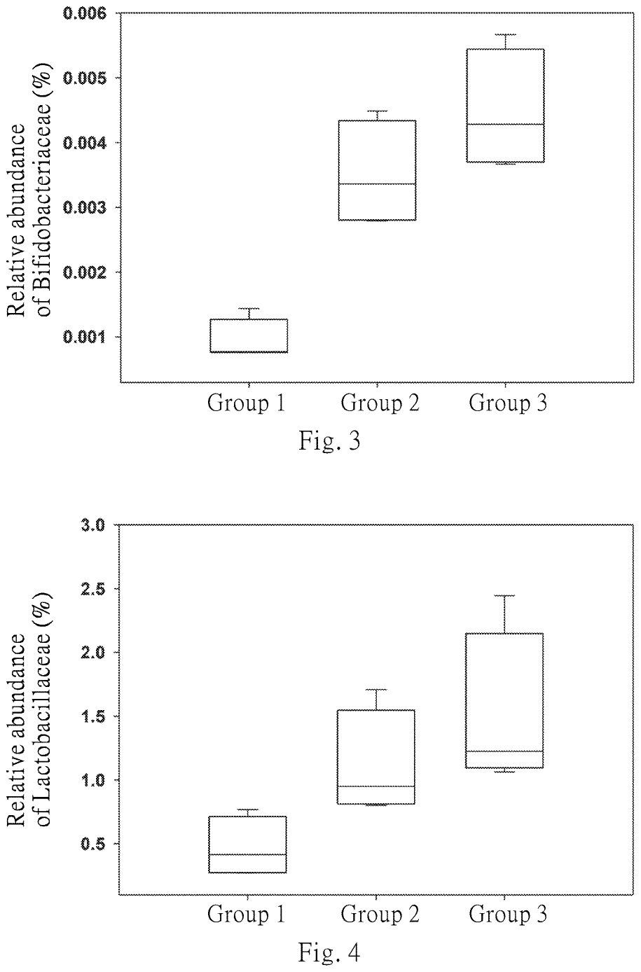

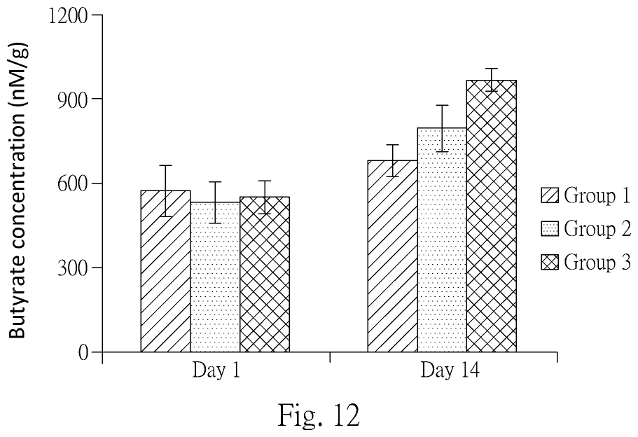

Feces of the mice in each group were collected on day 1 and day 14 of the experiment for next-generation sequencing (NGS) analysis, and the content of short-chain fatty acids (SCFAs) in the feces was determined by gas chromatography-mass spectrometry (GC-MS). The results are shown in FIG. 3 to FIG. 12.

The NGS analysis involved extracting fecal DNA from the fecal samples using a commercial kit (QIAamp Fast DNA Stool Mini Kit, Germany), followed by sequencing analysis.

The SCFA content analysis was carried out as follows. 30 mg of fecal sample was added with deionized water (300 μL), homogenized, and centrifuged. 150 μl of the supernatant was collected and acidified with 50 μL of 50% sulfuric acid. After that, 10 μL of 2-ethylbutyric acid and 400 μl of diethyl ether were added and thoroughly mixed. The resulting mixture was centrifuged at 9000 rpm and 4° C. for 10 minutes, and the ether layer was collected, dehydrated by adding anhydrous sodium sulfate, and then subjected to GC-MS analysis. The gas-phase ions of the short-chain fatty acids (including acetic acid, propionic acid, and butyric acid) in the samples were detected based on the mass-to-charge ratio.

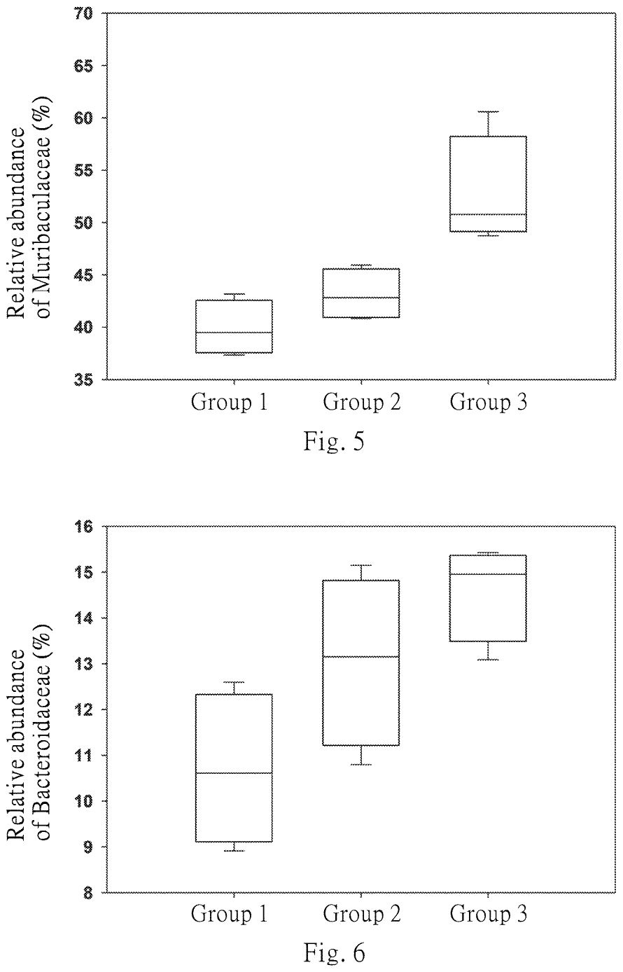

According to the results in FIG. 3 and FIG. 4, the proliferation rate of beneficial microbiota in the intestinal tract of the mice in each of group 2 and group 3 was significantly higher than that of group 1, wherein the beneficial microbiota included microbiota of the family Bifidobacteriaceae (including Bifidobacterium) and microbiota of the family Lactobacilluseae (including Lactobacillus acidophilus). Notably, the proliferation rate of the beneficial microbiota in the intestinal tract of the mice in group 3 was higher than that of group 2, indicating that a higher dose of Sarcodia suae polysaccharide is more effective in promoting the proliferation of the beneficial bacteria. Furthermore, the results in FIG. 5 and FIG. 6 show that the proliferation rate of intestinal commensal bacteria, such as those of the families Muribaculaceae and Bacteroidaceae, in the mice in each of group 2 and group 3 was significantly higher than that of group 1. Notably, the proliferation rate of the intestinal commensal microbiota in the mice in group 3 was higher than that of group 2, indicating that a higher dose of Sarcodia suae polysaccharide is more effective in increasing the population of the intestinal commensal microbiota.

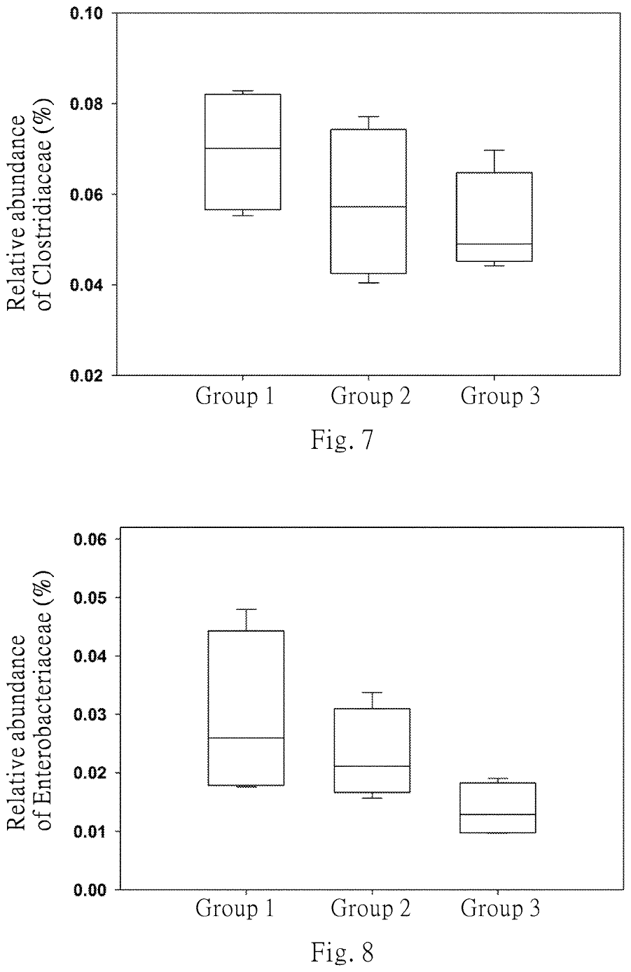

According to the results in FIG. 7 and FIG. 8, the relative abundance of harmful bacteria in the intestinal tract of the mice in each of group 2 and group 3 was lower than that of group 1, wherein the harmful bacteria included those of the family Clostridiaceae (including Clostridioides difficile) and those of the family Enterobacteriaceae (including Escherichia coli). Notably, the population of pathogenic microbiota in the intestinal tract of the mice in group 3 was significantly lower than that of group 2, indicating that a higher dose of Sarcodia suae polysaccharide exhibits a stronger inhibitory effect on the intestinal pathogenic bacteria.

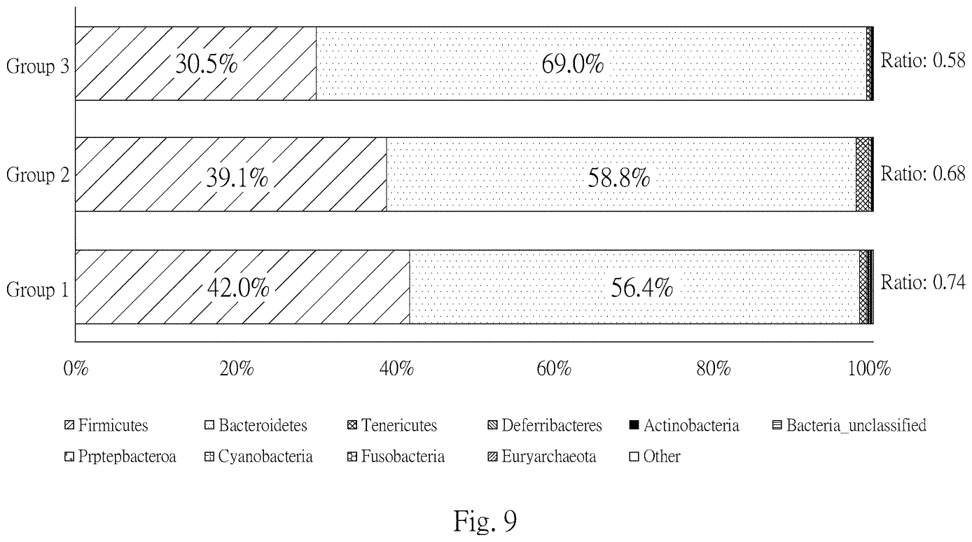

According to the results in FIG. 9, the relative abundance of microbiota of the family Bacteroidaceae in the intestinal tract of the mice in each of group 2 and group 3 was significantly increased, and this further confirms that the Sarcodia sua e polysaccharide disclosed in the present invention can promote the proliferation of beneficial intestinal microbiota. Moreover, given that microorganisms of the phylum Bacteroidetes are associated with the degradation and fermentation of polysaccharides and the production of such metabolites as propionate, and are capable of increasing the production of acetate and propionate, the results in FIG. 9 also demonstrate that the Sarcodia suae polysaccharide disclosed in the invention has the activities of improving intestinal metabolic health, resisting intestinal inflammation, and regulating metabolism in an individual.

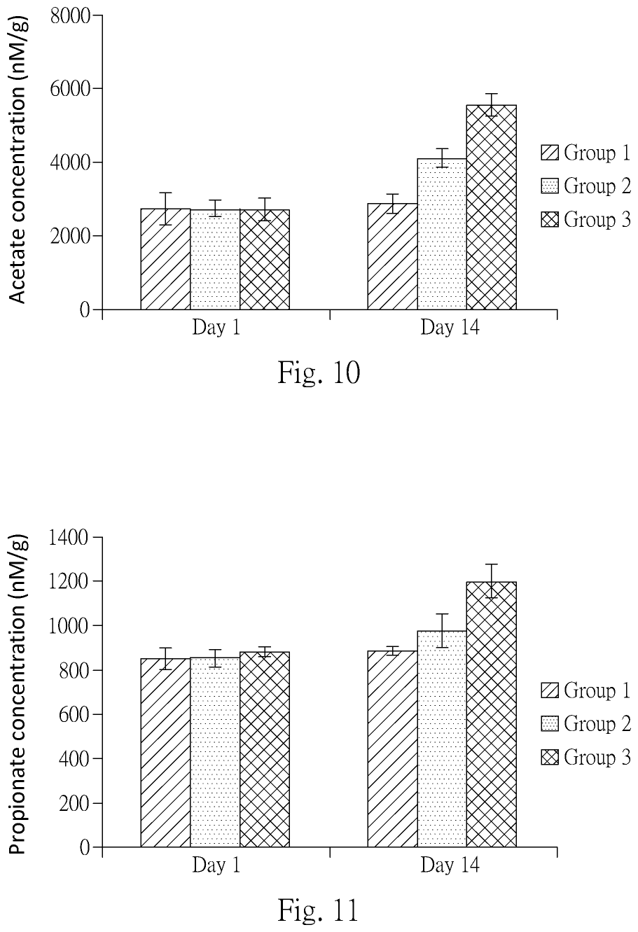

According to the results in FIG. 10 to FIG. 12, the concentrations of acetate, propionate, and butyrate in the intestinal tract of the mice in each of group 2 and group 3 were higher than those of group 1 on day 14 of the experiment. Notably, the concentrations of acetate, propionate, and butyrate in the intestinal tract of the mice in group 3 were higher than those of group 2. These results indicate that the Sarcodia suae polysaccharide disclosed in the present invention has the activity of increasing the production of short-chain fatty acids in the intestinal tract.

In summary, the results disclosed in this example demonstrate that the algal polysaccharide disclosed in the present invention has the activities of promoting the proliferation of beneficial intestinal microbiota, increasing the metabolic activities of intestinal commensal bacteria, inhibiting the growth of intestinal pathogenic bacteria, reducing the population of pathogenic microbiota, and increasing the production of short-chain fatty acids in the intestinal tract. That is to say, administering an effective amount of the algal polysaccharide or a composition containing the algal polysaccharide to an individual can maintain intestinal microbiota balance, promote intestinal barrier repair, and produce an anti-inflammatory effect, thereby improving or preventing metabolic diseases or diseases associated with intestinal dysbiosis and maintaining the individual's metabolic functions.

### Example 2: Anti-Allergic Activity Assay

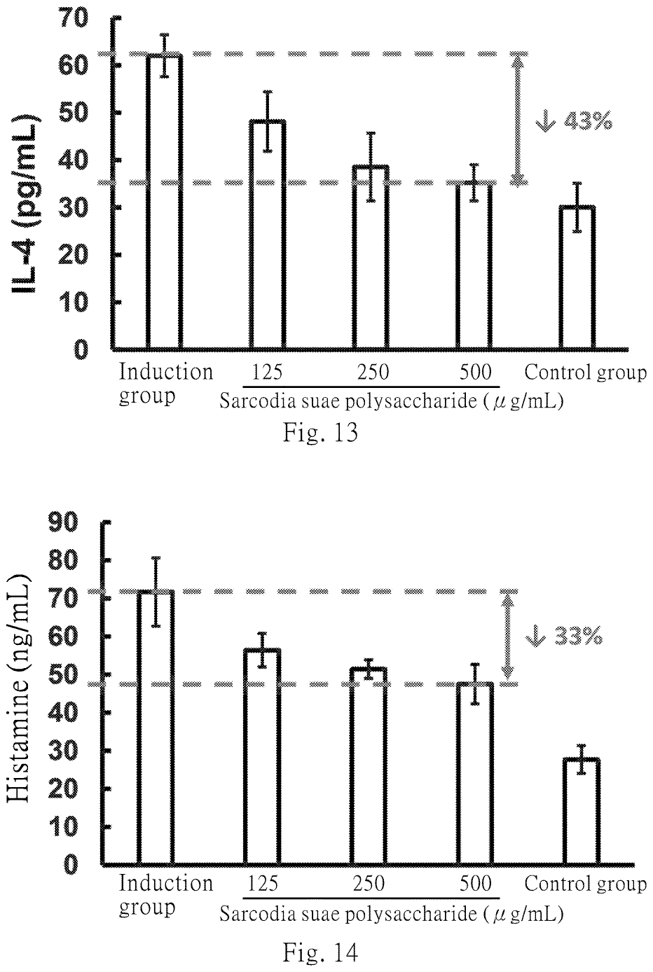

P815 mast cells were seeded in a 24-well culture plate at a concentration of 1×10⁶ cells/mL and cultured in an environment of 37° C. and 5% CO₂ for 24 hours. After that, Sarcodia suae polysaccharide of different concentrations (0, 125, 250, and 500 μg/mL) was added separately to co-culture with the P815 mast cells for 1 hour. Compound 48/80 (10 μg/mL) was then added, followed by incubation for 6 hours. The culture medium was collected, and the concentrations of IL-4 and histamine in the culture medium were detected using ELISA kits. The results are shown in FIG. 13 and FIG. 14. In this assay, P815 cells that were neither treated with Sarcodia suae polysaccharide nor induced were used as a control group, and P815 cells that were not treated with Sarcodia suae polysaccharide but were induced served as an induction group.

According to the results in FIG. 13 and FIG. 14, the concentrations of IL-4 and histamine in each of the cell groups treated with Sarcodia suae polysaccharide were significantly lower than those of the induction group. More specifically, in the cell group treated with 500 μg/mL Sarcodia suae polysaccharide, for example, the IL-4 concentration and the histamine concentration were decreased by 43% and 33%, respectively, compared with the induction group.

These results demonstrate that the Sarcodia suae polysaccharide disclosed in the present invention can reduce the production of IL-4 and histamine caused by the degranulation of mast cells, thereby achieving the effect of reducing allergic reactions. In other words, administering an effective amount of the algal polysaccharide disclosed in the invention to an individual can effectively improve or prevent the onset of allergic diseases.

### Example 3: Anti-Inflammatory Assay

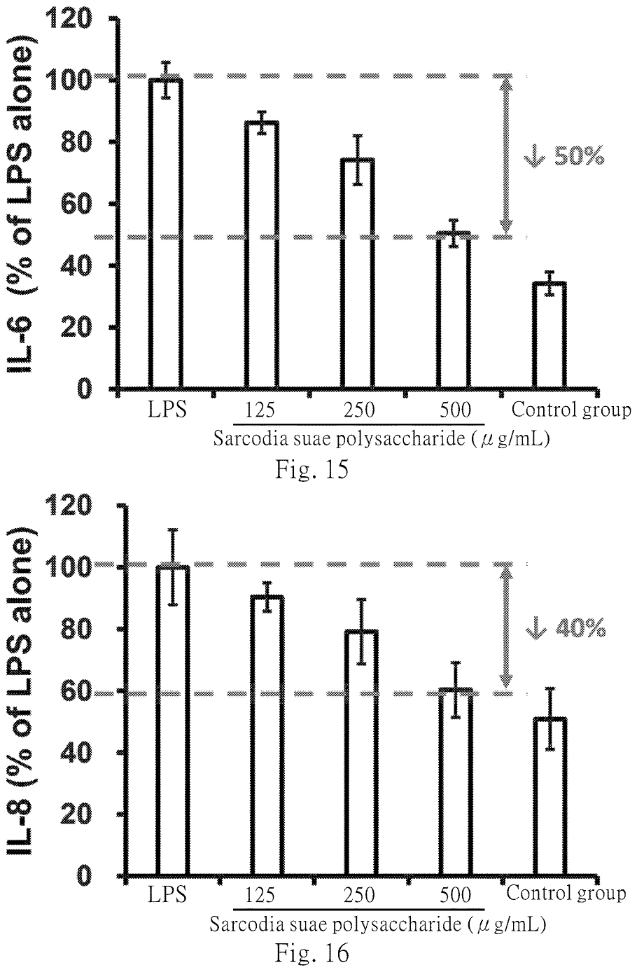

RAW 264.7 cells were seeded in a 96-well culture plate at a concentration of 2×10⁵ cells/mL. Sarcodia suae polysaccharide of different concentrations (0, 125, 250, and 500 μg/mL) was added separately, followed by the addition of LPS (1 μg/mL) and incubation for 24 hours. After the incubation, the culture medium was collected, and the concentrations of IL-6 and IL-8 in the culture medium were detected using ELISA kits. The results are shown in FIG. 15 and FIG. 16. In this assay, cells that were neither treated with Sarcodia suae polysaccharide nor induced with LPS were used as a control group, and cells that were not treated with Sarcodia suae polysaccharide but were induced with LPS served as an LPS group.

According to the results in FIG. 15 and FIG. 16, the concentrations of IL-6 and IL-8 in each of the cell groups treated with Sarcodia suae polysaccharide were significantly lower than those of the LPS group. More specifically, in the cell group treated with 500 μg/mL Sarcodia suae polysaccharide, for example, the IL-6 concentration and the IL-8 concentration were decreased by 50% and 40%, respectively, compared with the LPS group.

Studies have indicated that Malassezia, a fungus responsible for dandruff, can upregulate the expression of the inflammatory factor IL-8 and exhibits chemotaxis toward neutrophils and irritating fatty acids. Therefore, from the results in FIG. 15 and FIG. 16, which show that the Sarcodia suae polysaccharide disclosed in the present invention has the activity of reducing inflammatory factors, it can be inferred that the algal polysaccharide disclosed in the invention can be used to treat or improve inflammation or diseases associated therewith, and achieve the effects of improving dandruff or soothing the scalp by reducing the production of inflammatory factors.

### Example 4: Anti-UV Assay

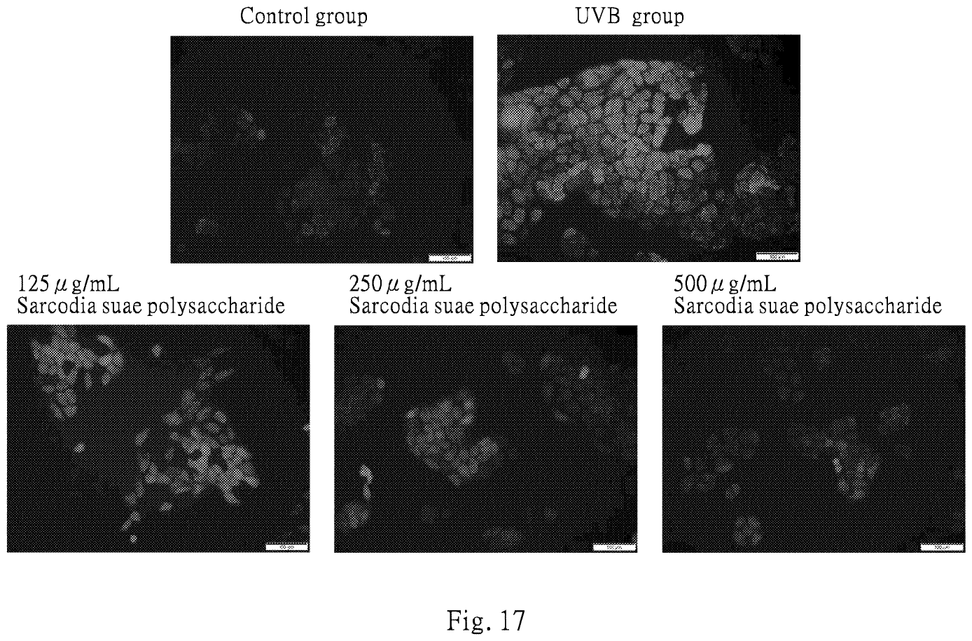

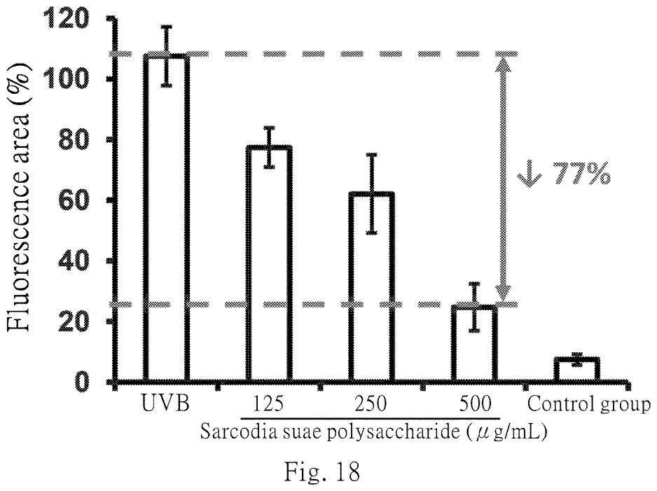

HaCaT cells were seeded in culture dishes at a concentration of 5×10⁴ cells/mL, and Sarcodia suae polysaccharide of different concentrations (0, 125, 250, and 500 μg/mL) was added separately to co-culture with the HaCaT cells. After the culture medium was removed from each cell group, the cells were irradiated with UVB (100 mJ/cm², 360 seconds), and then fluorescence detection and an intracellular ROS (reactive oxygen species) assay were performed on each cell group. The results are shown in FIG. 17 and FIG. 18. In this anti-UV assay, cells that were neither treated with Sarcodia suae polysaccharide nor irradiated with UVB were used as a control group, and cells that were not treated with Sarcodia suae polysaccharide but were irradiated with UVB served as a UVB group.

According to the results in FIG. 17 and FIG. 18, the fluorescence areas in each of the cell groups treated with Sarcodia suae polysaccharide—in particular the cell group treated with Sarcodia suae polysaccharide at a dose of 500 μg/mL—were significantly smaller than those of the UVB group. More specifically, in the cell group treated with 500 μg/mL Sarcodia suae polysaccharide, the fluorescence areas were reduced by 77% compared with the UVB group.

The results of this example demonstrate that the algal polysaccharide disclosed in the present invention has the activity of reducing the production of ROS caused by UVB irradiation and can therefore enhance the skin barrier function to achieve the effects of protecting skin from UV damage and resisting skin aging.

### Example 5: Wound Repair Assay

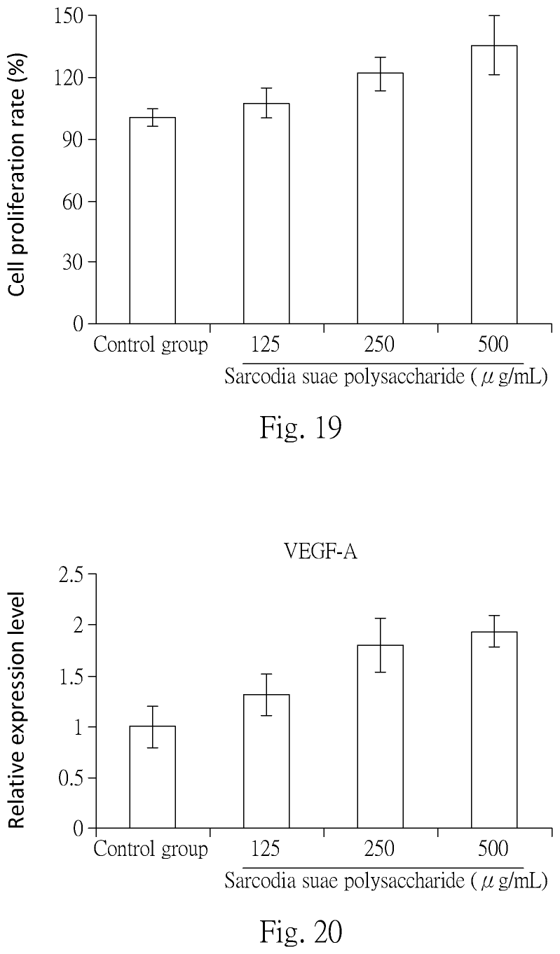

HaCaT cells were seeded in a 6-well culture plate at a concentration of 2×10⁵ cells/mL. After the cells attached to form a monolayer, a scratch was made in the cell layer to create a wound. Detached cells were removed with phosphate-buffered saline, and then the culture medium was replaced with culture media each containing Sarcodia suae polysaccharide at a different concentration (0, 125, 250, or 500 μg/mL) for further incubation. Twenty-four hours after scratching, 10 μL of CCK-8 reagent was added, followed by incubation for 2 hours. An ELISA reader was then used to measure the absorbance at a wavelength of 450 nm, and the relative cell proliferation rate of each experimental group was calculated, with the control group (whose culture medium contained 0 μg/mL Sarcodia suae polysaccharide) set as 100%. The results are shown in FIG. 19.

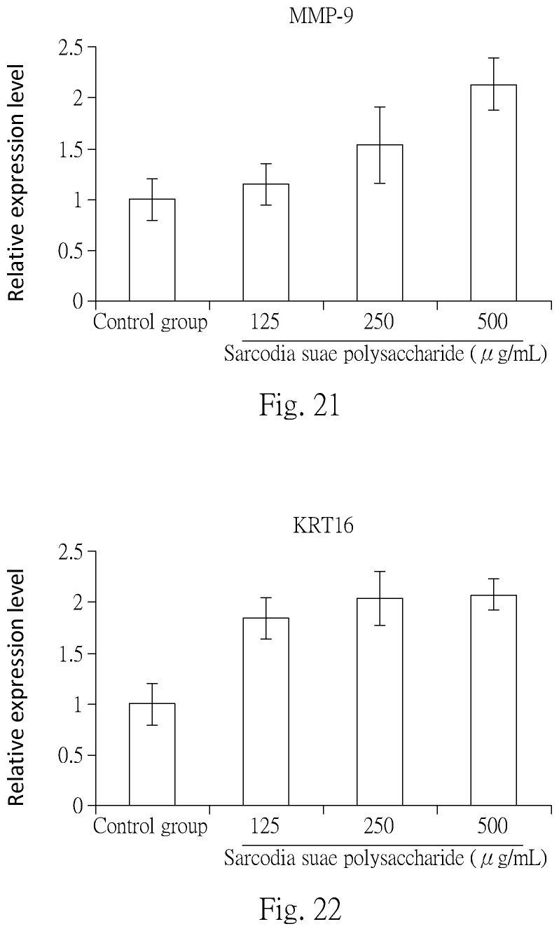

In addition, 24 hours after scratching, the HaCaT cells of each group were collected for detection of the expression levels of the MMP-9, VEGF-A, and KRT16 genes. The results are shown in FIG. 20 to FIG. 22.

It can be known from the results in FIG. 19 to FIG. 22 that the culture media containing Sarcodia suae polysaccharide significantly promoted the proliferation of HaCaT cells, accelerated the cell migration rate during cell repair, and increased the expression levels of the wound healing-related genes, namely MMP-9, VEGF-A, and KRT16. These results demonstrate that the Sarcodia suae polysaccharide disclosed in the present invention has the activity of activating epithelial cell repair and can therefore achieve the effects of promoting wound healing and accelerating wound repair.

What is claimed is:

1. A method for extracting an algal polysaccharide for whole-algae utilization, comprising:
a first-stage extraction process comprising: performing extraction on an algal raw material with a first extraction solvent to obtain a first extraction mixture;
a second-stage extraction process comprising: collecting a first precipitate from the first extraction mixture, and performing extraction on the first precipitate with a second extraction solvent to obtain a second extraction mixture; and
a third-stage extraction process comprising: collecting a second precipitate from the second extraction mixture, performing extraction on the second precipitate with a third extraction solvent to obtain a third extraction mixture, and filtering the third extraction mixture to obtain a third filtrate containing an algal polysaccharide;
wherein:
the second extraction solvent used in the second-stage extraction process is different from the first extraction solvent used in the first-stage extraction process and the third extraction solvent used in the third-stage extraction process;
each of the first-stage extraction process and the second-stage extraction process uses an extraction temperature of 10-40° C., and the third-stage extraction process uses an extraction temperature higher than the extraction temperatures of the first-stage extraction process and of the second-stage extraction process; and
the algal polysaccharide has a total sugar content of at least 80 (w/w) %, a water-soluble component of at least 70 (w/w) %, and a viscosity of 2200-2510 cps, and the algal polysaccharide has a monosaccharide composition comprising 56-63% galactose, 27-33% glucose, 5-6% xylose, and 3-5% mannose.

2. The method for extracting an algal polysaccharide for whole-algae utilization as claimed in claim 1, wherein the first-stage extraction process comprises:
step 101: obtaining the algal raw material;
step 102: performing extraction on the algal raw material with the first extraction solvent at an extraction temperature of 20-40° C. to obtain the first extraction mixture, wherein the first extraction solvent is water; and
step 103: filtering the first extraction mixture to obtain a first filtrate and the first precipitate, wherein the first precipitate contains phycobiliproteins.

3. The method for extracting an algal polysaccharide for whole-algae utilization as claimed in claim 1, wherein the second-stage extraction process comprises:
step 201: collecting the first precipitate;
step 202: performing extraction on the first precipitate with the second extraction solvent at an extraction temperature of 10-30° C. to obtain the second extraction mixture, wherein the second extraction solvent is ethanol at a concentration of 75-95%; and
step 203: filtering the second extraction mixture to obtain a second filtrate and the second precipitate, wherein the second precipitate contains phytochemicals.

4. The method for extracting an algal polysaccharide for whole-algae utilization as claimed in claim 1, wherein the third-stage extraction process comprises:
step 301: obtaining the second precipitate;
step 302: performing extraction on the second precipitate with the third extraction solvent at an extraction temperature of 90-100° C. to obtain the third extraction mixture, wherein the third extraction solvent is water; and
step 303: filtering the third extraction mixture to obtain the third filtrate and a third precipitate, wherein the third precipitate contains seaweed fiber.

5. A method for regulating intestinal microbiota with an algal polysaccharide, comprising administering an effective amount of the algal polysaccharide to an individual to regulate abundance of at least one microorganism in the individual's intestinal tract, wherein the algal polysaccharide is prepared by the method for extracting an algal polysaccharide for whole-algae utilization as claimed in claim 1.

6. The method for regulating intestinal microbiota with an algal polysaccharide as claimed in claim 5, wherein the microorganism is a beneficial bacterium classified under the family Bifidobacteriaceae, Lactobacilluseae, Muribaculaceae, or Bacteroidaceae, and the algal polysaccharide is capable of increasing a population of the beneficial bacterium.

7. The method for regulating intestinal microbiota with an algal polysaccharide as claimed in claim 5, wherein the microorganism is a harmful bacterium classified under the family Clostridiaceae or Enterobacteriaceae, and the algal polysaccharide is capable of inhibiting growth of the harmful bacterium.

8. The method for regulating intestinal microbiota with an algal polysaccharide as claimed in claim 5, wherein the algal polysaccharide is capable of increasing short-chain fatty acid levels in the individual's intestinal tract.
